(19) United States
(12) Patent Application Publication
(10) Pub. No.: US 2008/0091179 A1
(43) Pub. Date: Apr. 17, 2008

(54) COMPACT, HANDHELD DEVICE FOR HOME-BASED ACNE TREATMENT

(75) Inventors: Anthony J. Durkin, Irvine, CA (US); Dilip Y. Paithankar, Natick, MA (US); Yacov Domankevitz, Newton, MA (US); James C. Hsia, Weston, MA (US); Jayant D. Bhawalkar, Brighton, MA (US); Paul R. Lucchese, Sudbury, MA (US)

Correspondence Address: PROSKAUER ROSE LLP, ONE INTERNATIONAL PLACE, BOSTON, MA, 02110, US

(73) Assignee: Candela Corporation, Wayland, MA (US)

(21) Appl. No.: 11/777,815

(22) Filed: Jul. 13, 2007

Related U.S. Application Data
(63) Continuation-in-part of application No. 10/012,241, filed on Nov. 5, 2001.
Application No. 10/012,241 is a continuation-in-part of application No. 09/731,496, filed on Dec. 7, 2000, now Pat. No. 6,743,222.
(60) Provisional application No. 60/830,641, filed on Jul. 13, 2006.
(60) Provisional application No. 60/170,244, filed on Dec. 10, 1999.

(51) Int. Cl. A61B 18/18

(52) U.S. Cl. 606/9; 604/20

(57) ABSTRACT

A compact, handheld device can be used to treat a sebaceous follicle disorder in a preselected dermal region of mammalian skin. A treatment can ameliorate at least one symptom of a lesion characteristic of the disorder.

### RELATED APPLICATION

This application is a continuation-in-part of U.S. application Ser. No. 10/012,241, filed Nov. 5, 2001, which is a continuation-in-part of U.S. application Ser. No. 09/731,496, filed Dec. 7, 2000, now U.S. Pat. No. 6,743,222, and claims the benefit of and priority to U.S. Application Ser. No. 60/170,244, filed Dec. 10, 1999, and U.S. Application Ser. No. 60/830,641, filed Jul. 13, 2006, the disclosures of each of which are incorporated by reference herein in their entirety.

### GOVERNMENT RIGHTS

This work was supported, in part, by Federal Grant No. 1-R43-AR 46938-01, awarded under the Small Business Innovation Research Program of the Department of Health and Human Services, Public Health Service. The Government may have certain rights in the invention

### FIELD OF THE INVENTION

The invention relates generally to a method of treating a mammalian skin disorder associated with sebaceous follicles. More particularly, the invention relates to a method of treating acne in a mammal using a compact, handheld device.

### BACKGROUND OF THE INVENTION

There are a variety of disorders associated with sebaceous follicles (also referred to herein as sebaceous follicle disorders) known to afflict mammals, in particular, humans. The disorders usually are associated with aberrations (for example, structural or functional aberrations) of the sebaceous follicles. In humans, sebaceous follicles, although present over most of the body surface, usually are largest and most dense on the face, chest and upper back. Accordingly, sebaceous follicle disorders predominantly affect these areas of the human body.

Probably the most pervasive sebaceous follicle disorder in the United States is acne, which affects between 40 to 50 million individuals in the United States (White GM, (1998) “Recent findings in the epidemiologic evidence, classification, and subtypes of acne vulgaris,” J. AM. ACAD. DERMATOL. 39(2 Pt 3): S34-7). Acne occurs with greatest frequency in individuals between the ages of 15 and 18 years, but may begin at virtually any age and can persist into adulthood. In the 12- to 17-year old range, the incidence has been reported to be 25% (Strauss JS, (1982) “Skin care and incidence of skin disease in adolescence,” CURR. MED. RES. OPIN. 7(Suppl 2):33-45). Acne is a disorder characterized by inflammatory, follicular, papular and/or pustular eruptions involving the sebaceous follicles (Stedman's Medical Dictionary, 26^{th} edition, (1995) Williams & Wilkins). Although there are a variety of disorders that fall within the acne family, for example, acne conglobata, acne rosacea, and acne vulgaris, acne vulgaris probably is the most notable and commonly known form of acne. Because acne vulgaris can lead to permanent scarring, for example, facial scarring, this form of acne can have profound and long-lasting psychological effects on an afflicted individual. Furthermore, pustule formation and scarring can occur at an age when the potential impact on an individual is greatest. As a result, enormous amounts of money (i.e., on the order of billions of dollars) are spent annually in the United States on various topical and systemic acne treatments. These treatments often are employed without the guidance or supervision of a physician.

Acne vulgaris typically results from a blockage of the opening of the sebaceous follicle. It is believed that both (i) the amount of sebum, a lipid, keratin and cellular debris containing fluid, produced and secreted by the sebaceous glands and (ii) bacteria, namely, Propionibacterium acnes (P. acnes) which metabolize lipids in the sebum, play a role in formation and development of acne vulgaris. The basic lesion of acne vulgaris is referred to as a comedo, a distension of the sebaceous follicle caused by sebum and keratinous debris. Formation of a comedo usually begins with defective keratinization of the follicular duct, resulting in abnormally adherent epithelial cells and plugging of the duct. When sebum production continues unabated, the plugged follicular duct distends. A blackhead (or open comedo) occurs when a plug comprising a melanin containing blackened mass of epithelial debris pushes up to opening of the follicular duct at the skin surface. A whitehead (or closed comedo) occurs when the follicle opening becomes very tightly closed and the material behind the closure ruptures the follicle causing a low-grade dermal inflammatory reaction. Accordingly, some comedones, for example, in acne vulgaris, evolve into inflammatory papules, pustules, nodules, or chronic granulomatous lesions. Proliferation of P. acnes can result in the production of inflammatory compounds, eventually resulting in neutrophil chemotaxis (Skyes and Webster (1994) DRUGS 48: 59-70).

At present, acne patients may receive years of chronic topical or systemic treatments. Current treatment options include, for example, the use of topical anti-inflammatory agents, antibiotics and peeling agents, oral antibiotics, topical and oral retinoids, and hormonal agonists and antagonists. Topical agents include, for example, retinoic acid, benzoyl peroxide, and salicylic acid (Harrison's Principles of Internal Medicine, 14^{th} edition, (1998) Fauci et al., eds. McGraw-Hill). Useful topical antibiotics include, for example, clindamycin, erythromycin, and tetracycline and useful systemic antibiotics include, for example, erythromycin, tetracycline, and sulphanilamides (see, for example, U.S. Pat. Nos. 5,910,493 and 5,674,539). Administration of the systemic retinoid, isotretinion, has demonstrated some success in the treatment of acne (Harrison's Principles of Internal Medicine, 14^{th} edition, (1998) Fauci et al., eds. McGraw-Hill). Studies indicate that this drug decreases sebaceous gland size, decreases the rate of sebum production and/or secretion, and causes ductal epithelial cells to be less adherent, thereby preventing precursor lesions of acne vulgaris (Skyes and Webster (1994) supra). Side-effects, however, include dry mouth and skin, itching, small red spots in the skin, and eye irritation. A significant concern about oral retinoids is their possible teratogenicity (Turkington and Dover (1996) SKIN DEEP: AN A-Z OF SKIN DISORDERS, TREATMENT AND HEALTH FACTS ON FILE, Inc., New York, page 9). In addition, a variety of hormone-related, for example, corticosteroid anti-inflammatory therapies have been developed for the treatment of acne. These therapies can be expensive and most are associated with deleterious systemic or localized side-effects (Strauss (1982) “Skin care and incidence of skin disease in adolescence,” CURR. MED. RES. OPIN. 7(Suppl 2): 33-45).

Because the foregoing therapies generally do not affect the structure and/or function of sebaceous follicles associated with the disease, the treatments remain non-curative. In other words, the disorder may recur after cessation of therapy. The result can be years of chronic therapy, and potential scarring for the patient, and enormous associated health care costs.

In recent years, a variety of laser-based methodologies for treating acne have been developed. The methods generally involve the combination of laser radiation and either an exogenous or endogenous chromophore present in the target tissue so that the laser light is absorbed preferentially in the target tissue causing morphological changes to the sebaceous follicle and/or causing a reduction of sebum production. For example, U.S. Pat. No. 5,817,089 describes a laser-based method for treating acne requiring topical application of a light absorbing chromophore, for example, micron graphite particles dispersed in mineral oil, onto skin needing such treatment. Similarly, U.S. Pat. No. 5,304,170 also describes a laser-based method for treating acne in which target cells contain greater amounts of a light absorbing chromophore, for example, the carotenoid β-carotene, relative to lesser or non-pigmented surrounding cells. In the chromophore based methods it can be difficult to get sufficient chromophore in the target region to elicit selective tissue damage and the method may still damage the outer layers of the skin resulting in scarring.

### SUMMARY OF THE INVENTION

The invention features, in one embodiment, an apparatus for treating a sebaceous follicle disorder of mammalian skin, for example, human skin. The apparatus can be a portable, handheld device. The apparatus can provide a sub-surface treatment method in which the regions of skin dermis containing sebaceous follicles are treated and the overlying regions of the epidermis/dermis and the underlying portions of the dermis are spared from thermal damage. The invention offers numerous advantages over existing treatment protocols. For example, the method provides a long lasting treatment which persists long after treatment has ceased. Furthermore, the method minimizes trauma and scar formation at the skin surface, reduces side-effects, such as, pain, erythema, edema, and blistering, which can result from other treatments, and can also minimize pigmentary disturbances of the skin. Light can be applied to the skin to induce a thermal change to the portion of the dermis where a sebaceous follicle resides. This heating can result in the destruction of the sebaceous follicle or the sebaceous gland associated with the follicle, cause structural changes in the follicle to reduce the likelihood of blockage, reduce the level of sebum production, and/or improve the appearance of the sebaceous follicle. A cooling step can serve to preserve the epidermis and the dermis overlaying the sebaceous gland containing region of the skin. The cooling step can be performed prior to, contemporaneous with, or after application of the energy to the target region, or alternatively the cooling can result from a combination of such cooling steps.

In one aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion. The method also includes delivering the radiation to the preselected dermal region of skin to ameliorate the lesion associated with the sebaceous follicle disorder while keeping the temperature of a region of the skin above the preselected dermal region below about 60° C. during application of the energy.

In another aspect, the invention features a compact, handheld device for treating a sebaceous follicle disorder. The compact, handheld device includes a handheld housing, a power source associated with the handheld housing, a tungsten lamp, an activator, a cooling device, and a reflector. The tungsten lamp is disposed at a first end of the handheld housing and receives power from the power source to generate radiation. The activator is associated with the handheld housing and activates the tungsten lamp to generate the radiation. The cooling device keeps the temperature of a region of the skin near the sebaceous follicle disorder below about 60° C. during application of the radiation. The reflector is disposed proximally to the tungsten lamp, receives at least a portion of the radiation generated by the tungsten lamp, and directs at least a portion of the radiation to a target region of skin to treat the sebaceous follicle disorder.

In still another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion, the compact, handheld device including a vacuum chamber transparent to the radiation. The method also includes drawing the preselected dermal region against a skin contacting element of the vacuum chamber and delivering the radiation to the preselected dermal region of skin to ameliorate the lesion associated with the sebaceous follicle disorder.

In yet another aspect, the invention features a compact, handheld device for treating a sebaceous follicle disorder. The compact, handheld device includes a handheld housing, a power source associated with the handheld housing, a radiation source, a vacuum chamber, and an activator. The radiation source is disposed at a first end of the handheld housing and receives power from the power source to generate radiation. The vacuum chamber is transparent to the radiation and draws the preselected dermal region against a skin contacting element of the vacuum chamber. The activator is associated with the handheld housing, activates the radiation source to generate the radiation, and at least a portion of the radiation is directed through the vacuum chamber to a target region of skin to treat the sebaceous follicle disorder.

In another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion, and stretching the preselected dermal region of skin to enhance the penetration of the skin by the radiation. The method also includes delivering the radiation to the preselected dermal region of skin to ameliorate the lesion associated with the sebaceous follicle disorder while keeping the temperature of the region of the skin above the preselected dermal region below about 60° C. during application of the energy.

In still another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion, the compact, handheld device having a diffusing unit to improve bodily safety during exposure by scattering the radiation. The method also includes delivering the scattered radiation to the preselected dermal region of skin to ameliorate the lesion associated with the sebaceous follicle disorder while keeping the temperature of the region of the skin above the preselected dermal region below about 60° C. during application of the energy.

In yet another aspect, the invention features a compact, handheld device for treating a sebaceous follicle disorder. The compact, handheld device includes a handheld housing, a power source associated with the handheld housing, a radiation source, a diffusing unit, and an activator. The radiation source is posed at a first end of the handheld housing and receives power from the power source to generate radiation. The diffusing unit improves bodily safety during exposure by scattering the radiation. The activator is associated with the handheld housing and activates the radiation source to generate the radiation, and at least a portion of the radiation is directed through the diffusing unit to a target region of skin to treat the sebaceous follicle disorder.

In another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion, the compact, handheld device has an integrating sphere to improve bodily safety during exposure by at least one of scattering and multiple internal reflection of the radiation. The method also includes delivering the scattered radiation to the preselected dermal region of skin to ameliorate the lesion associated with the sebaceous follicle disorder while keeping the temperature of the region of the skin above the preselected dermal region below about 60° C. during application of the energy.

In still another aspect, the invention features a compact, handheld device for treating a sebaceous follicle disorder. The compact, handheld device includes a handheld housing, a power source associated with the handheld housing, a radiation source, an integrating sphere, and an activator. The radiation source is disposed at a first end of the handheld housing and receives power from the power source to generate radiation. The integrating sphere improves bodily safety during exposure by at least one of scattering and multiple internal reflection of the radiation. The activator is associated with the handheld housing for activating the radiation source to generate the radiation, at least a portion of the radiation to be directed through the integrating sphere to a target region of skin to treat the sebaceous follicle disorder.

In yet another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion. The method also includes delivering the radiation to the preselected dermal region of skin to ameliorate the lesion associated with the sebaceous follicle disorder and applying a medicament for treating the sebaceous follicle disorder at least one of before, during, and after treatment.

In another aspect, the invention features a kit for treating a sebaceous follicle disorder in a preselected dermal region of mammalian skin, the preselected dermal region having at least one lesion characteristic of the disorder disposed therein. The kit includes a compact, handheld device generating radiation having energy in an amount sufficient to ameliorate the lesion. The kit also includes a medicament for treating the sebaceous follicle disorder at least one of before, during, and after applying radiation generated by the compact, handheld device.

In still another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion and applying a heat retaining element to a surface of the skin, the heat retaining element at least partially transparent to the radiation. The method also includes delivering a first portion of the radiation through the heat retaining element to the preselected dermal region of skin to ameliorate the lesion associated with the sebaceous follicle disorder and delivering a second portion of the radiation to the heat retaining element to convert the second portion of the radiation to thermal energy within the heat retaining element, which is subsequently conducted to the preselected dermal region of skin to facilitate amelioration of the lesion associated with the sebaceous follicle disorder.

In yet another aspect, the invention features a method of treating a sebaceous follicle disorder in a preselected dermal region of mammalian skin, the preselected dermal region having at least one lesion characteristic of the disorder disposed therein. The method includes providing a compact, handheld device generating radiation having energy in an amount sufficient to ameliorate the lesion, the compact, handheld device having a skin contacting portion adapted for at least one of absorbing and releasing heat from the skin, the skin contacting portion substantially transparent to the radiation. The method also includes contacting the preselected dermal region with the skin contacting portion and delivering the radiation through the skin contacting portion to the preselected dermal region of skin to ameliorate the lesion associated with the sebaceous follicle disorder.

In other examples, any of the aspects above, or any apparatus or method described herein, can include one or more of the following features.

In various embodiments, the method includes keeping the temperature of the region of the skin above the preselected dermal region below about 60° C. during application of the energy. In one embodiment, the method can include heating the skin contacting portion to a temperature above about 32° C. before contacting the preselected dermal region. In another embodiment, the method can include cooling the skin contacting portion to a temperature below about 32° C. before contacting the preselected dermal region.

In some embodiments, the radiation has at least one wavelength between about 1,200 nm and about 1,800 nm. In one embodiment, the radiation has at least one wavelength preferentially absorbed by fat relative to water. The at least one wavelength can be between about 1,190 nm and about 1230 nm or between about 1,700 nm and about 1,760 nm. The radiation can have a power density below about 10 W/cm². The compact, handheld device includes a first laser diode doped to provide a first radiation having at least one wavelength preferentially absorbed by fat. The compact, handheld device can include a second laser diode doped to provide a second radiation having at least one wavelength preferentially absorbed by water. The compact, handheld device can include a memory medium programmed to activate the device for a preselected duration of time. The radiation can be delivered to the preselected dermal region of skin for about 100 μs to about 200 s.

In certain embodiments, the radiation ameliorates a lesion associated with the sebaceous follicle disorder. In one embodiment, the power source includes a battery. The power source can be disposed inside the handheld housing. The compact, handheld device can include an interlock switch to ensure radiation is only delivered when the device is in contact with the skin. The compact, handheld device can include a timing circuit configured to activate the emitter for a preselected duration of time. The compact, handheld device can include an emitter portion having a filter to select a portion of the radiation to treat the sebaceous follicle disorder. The reflector can includes a coating of an optically active material to enhance or filter emission of the radiation in a specified spectral window. The target region of skin can be greater than about 1 cm in diameter.

Other aspects and advantages of the invention will become apparent from the following drawings, detailed description, and claims, all of which illustrate the principles of the invention, by way of example only.

### BRIEF DESCRIPTION OF THE DRAWINGS

The foregoing and other objects, features and advantages of the invention will become apparent from the following description of preferred embodiments of the invention, as illustrated in the accompanying drawings. The drawings are not necessarily to scale, with emphasis instead being placed on illustrating the principles of the invention.

FIG. 1 is a schematic representation of a vertical cross section of a sebaceous follicle disposed within mammalian skin.

FIG. 2 is a schematic representation of an apparatus including a radiation source and delivery system useful in the practice of the invention.

FIG. 3 is a schematic representation of an exemplary compact, handheld device for treating a sebaceous follicle disposed within mammalian skin.

FIG. 4 is a sectional view of the compact, handheld device.

FIG. 5 is a schematic representation of another exemplary device for treating a sebaceous follicle disposed within mammalian skin.

FIG. 6 is a schematic representation of an end portion of the compact, handheld device including a diffusing optic and an interlock mechanism.

FIG. 7 is a schematic representation of an exemplary hand set of a delivery system in which coherent radiation and cryogen spray are applied to the same region of the skin surface.

FIG. 8 is a schematic representation of an exemplary timing diagram showing exemplary heating and cooling phases useful in the practice of the invention.

FIG. 9A is a schematic representation of an exemplary compact, handheld device.

FIG. 9B is a schematic representation of an end portion of another exemplary compact, handheld device.

FIG. 10 is a graphical representation of the output of an exemplary tungsten filament lamp.

FIG. 11 is a schematic representation of a device for compressing the skin.

### DESCRIPTION OF THE INVENTION

A sebaceous follicle or a sebaceous follicle disorder can be treated while at the same time preventing or minimizing damage to skin tissue surrounding sebaceous follicles afflicted with the disorder. In particular, sebaceous follicles and dermal regions containing sebaceous follicles can be targeted for heat injury whereas the underlying dermal and overlaying dermal and epidermal regions can be protected from thermal injury. The underlying dermal regions are protected from thermal injury because, by selection of appropriate parameters, it is possible to limit the penetration depth of the heating energy applied to the region. Accordingly, by choice of appropriate parameters, it is possible to heat skin tissue to a preselected depth thereby sparing the underlying tissue from thermal injury. The overlaying dermal and epidermal regions can be protected from thermal injury by selection of appropriate parameters. For example, a wavelength and/or energy can be chosen to minimize absorption by overlaying dermal and epidermal regions and maximize absorption by target chromophores. In certain embodiments, the overlaying dermal and epidermal regions can be protected from thermal injury by appropriate surface cooling. Accordingly, by choice of appropriate heating and/or cooling parameters, it is possible for the skilled artisan to induce thermal injury to a specific target zone within the dermis of the skin.

Thermal injury can be induced by applying energy in the form of light to a target region of skin. The energy is applied in an amount and for a time sufficient to induce thermal damage to a portion of the skin containing a sebaceous follicle. A treatment can be prophylactic or can be performed to ameliorate one or more symptoms or lesions associated with a sebaceous follicle disorder. A treatment can result in one or more of the following: reducing or eliminating the production of sebum in a sebaceous follicle, reducing or eliminating bacteria in a sebaceous follicle, altering the structure of a sebaceous follicle (e.g., increasing or decreasing the internal diameter of the sebaceous follicle), unplugging a blockage from a sebaceous follicle, and preventing blockage of a sebaceous follicle. As a result, a treatment can ameliorate a skin lesion associated with a sebaceous follicle disorder while at the same time preserving the surface of the skin exposed to the heating energy.

Exemplary sebaceous follicle disorders include, but are not limited to, acne, acne vulgaris, acne rosacea, acne conglobata, seborrhea, sebaceous adenoma, and sebaceous gland hyperplasia. Sebaceous follicle disorders, for example, acne vulgaris and seborrhea, sometimes are associated with the overproduction of sebum. For example, in acne vulgaris, the level of sebum production by sebaceous glands has been correlated with the severity of the disorder (Leyden (1995) J. AM. ACAD. DERM. 32: S15-25). Accordingly, in an exemplary embodiment, a treatment decreases or even eliminates sebum production by sebaceous glands of sebaceous follicles relative to untreated sebaceous follicles. In some embodiments, a treatment can increase the size of the opening of the sebaceous follicle, in the proximity of the infundibulum, thereby affecting sebum flow and/or minimizing the likelihood of blockage of the sebaceous follicle. Furthermore, a treatment may destroy or inactivate the sebaceous follicle to eliminate sebum production in that follicle. A treatment can also be a combination treatment (e.g., an acne treatment and a hair removal treatment).

The treatment can reduce the size of one or more lesions, for example, comedones in the case of acne vulgaris, disposed within the preselected region. Furthermore, the treatment can also reduce the number or density of the lesions disposed within the preselected region. In cases in which skin inflammation can be associated with the lesion, for example, in severe cases of acne vulgaris and acne conglobata, the treatment may reduce inflammation associated with the lesion. The benefit of treatment, for example, reduction in the number of or elimination of skin lesions, may become apparent days to weeks after the treatment. Furthermore, it is contemplated that in certain cases, e.g., severe cases, of sebaceous follicle disorders, multiple rounds of treatment, for example, two, three, four, five, six, seven, eight, nine, ten, or more separate rounds of treatment, may be required to treat an individual satisfactorily. In certain embodiments, a compact, handheld device can be used for home based treatments.

It is contemplated that, based upon choice of appropriate cooling and/or heat energy parameters, it is possible to create thermally induced changes of sebaceous follicles in the absence of an exogenous energy absorbing material. However, under some circumstances, optimal treatment may be facilitated by applying to the preselected region prior to exposure to the radiation, a light absorbing material, for example, a chromophore photoexcited by the radiation. The radiation absorbing material may be administered systemically to the mammal or applied topically to the preselected region prior to exposure to the radiation.

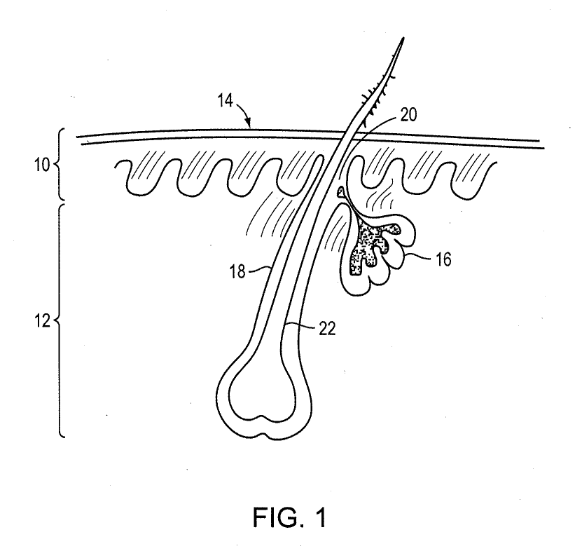

FIG. 1 is a schematic illustration of a cross-sectional view of a sebaceous follicle disposed within human skin. Skin is comprised primarily of two layers in which the top layer of skin, known as the epidermis 10, is supported by a layer known as the dermis 12. The epidermis 10, has an exposed surface 14. In human skin, epidermis 10 extends to a depth of about 60-100 microns from skin surface 14 whereas the underlying dermis 12 extends to a depth of about 4 to 5 millimeters from the skin surface 14. Furthermore, in skin, dermis 12 is supported by or is disposed upon a layer of subcutaneous fat (not shown). Dermis 12 is primarily acellular and comprises primarily water, collagen, and glycosaminoglycans. Water is believed to constitute approximately 60-80 percent of the total weight of the dermis. As shown, sebaceous gland 16 is in fluid flow communication with a hair duct 18. As a result, sebum produced by the sebaceous gland 16 flows into the hair duct 18. The upper portion of hair duct 18 which receives sebum from sebaceous gland 16 is referred to as the infundibulum 20. Hair shaft 22 is disposed within hair duct 18 and extends beyond the surface of the skin 14. Sebaceous glands usually are located at depths ranging from about 200 to about 1000 microns from the skin surface (Conontagna et al. (1992) in “ATLAS OF NORMAL HUMAN SKIN” by Springer Verlag, New York, N.Y.).

At birth, sebaceous follicles typically contain a small hair, a follicular orifice lined with epithelial cells, and a sebaceous gland. The outer layer of the sebaceous gland lobule is composed of undifferentiated hormonally responsive cells. In response to androgens, these cells, called sebocytes, divide and differentiate. Lipids accumulate and the cells enlarge and rupture, releasing their contents into the hair duct. Sebum, the product of the sebaceous gland, is composed of lipids and cellular debris combined with keratin and microorganisms, including the bacterium P. acnes (Sykes and Webster (1994) supra). Sebaceous glands and the sebum they produce have no proven function in humans, and in fact the skin of young children does not appear to be negatively affected by the almost lack of sebum (Staruss et al. (1992), J. INVEST. DERM., 67:90-97, and Stewart, M. E., (1992) SEMINAR. DERM. 11, 100-105).

As used herein, the term “sebaceous follicle” refers to any structure disposed within mammalian, particularly, human, skin, which comprises a hair follicle, also referred to herein as a hair duct, attached to and in fluid flow communication with a sebaceous gland. As a result, sebum produced by the sebaceous gland flows into the hair follicle. The sebaceous follicle optionally may include a hair shaft disposed within the hair follicle. The upper portion of the hair follicle into which sebum is released from the sebaceous gland is referred to as the infundibulum or a pore.

As used herein, the term “sebaceous follicle disorder” refers to any disorder of mammalian skin, in particular, human skin, that is associated with a sebaceous follicle. Sebaceous follicle disorders can result from an over production of sebum by a sebaceous gland of a sebaceous follicle and/or reduction or blockage of sebum flow in the infundibulum of the sebaceous follicle. Exemplary sebaceous gland disorders include, for example, acne, for example, acne vulgaris, acne rosacea, and acne conglobata, seborrhea, sebaceous adenoma and sebaceous gland hyperplasia.

As used herein, the term “lesion characteristic of the disorder” refers to any skin lesion associated with the sebaceous follicle disorder. For example, lesions associated with acne may include, without limitation, papules and pustules, and skin inflammation associated with the papules and pustules. In addition, specific lesions of acne conglobata include cystic lesions, abscesses and communicating sinuses, whereas specific lesions of acne vulgaris include comedones, cysts, papules and pustules on an inflammatory base. Lesions associated with seborrhea include, without limitation, dermatitis and eczema.

As used herein, the term “ameliorate a lesion” refers to a decrease in the size of a sebaceous follicle disorder-associated lesion and/or density of sebaceous follicle disorder-associated lesions in a preselected region, and can also include a decrease in skin-inflammation associated with the sebaceous follicle disorder.

As used herein, the terms “thermal change” or “thermal injury” with reference to sebaceous follicles refers to any change, for example, structural change and/or functional change, to the sebaceous follicle which ameliorates one or more lesions associated with the sebaceous follicle disorder. For example, sebum over-production can be a factor associated with certain sebaceous follicle disorders. Accordingly, a treatment can reduce sebaceous gland size and/or sebum production in the area afflicted with the disorder. Reduction in sebum production can occur when sebum producing cells disposed within the sebaceous glands are destroyed and thus inactivated, or when their sebum producing activity is reduced. Furthermore, a treatment can result in morphological changes to the sebaceous follicle, for example, increasing the diameter of the follicle, e.g., to minimize the likelihood of plug formation, or decreasing the diameter of the follicle, e.g., to improve the appearance of the follicle.

For example, by increasing the diameter of a follicle, the chance of plug formation is reduced so that any sebum produced by the sebaceous gland can still flow out of the sebaceous follicle. The changes are thermally induced and may result from the temperature-induced cell death and/or protein denaturation. Accordingly, an objective of a treatment can be to elevate the temperature of the dermal region containing sebaceous glands and more specifically the sebaceous gland to a level and for a time sufficient to cause cell death and/or protein denaturation.

By decreasing the diameter of a follicle, undesirable activity of an enlarged follicle can be suppressed or the appearance of a sebaceous follicle can be improved. In one embodiment, the diameter of the follicle can be decreased by treating and shrinking the sebaceous gland. In another embodiment, the diameter of the follicle can be decreased by treating the infundibulum or tissue surrounding the infundibulum. For example, by stimulating the production of collagen and/or the extracellular matrix of the skin, new tissue growth can be stimulated that causes the diameter of the follicle to decrease.

In one embodiment, the treatment radiation can partially denature collagen fibers in the target area. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target area can change and/or improve the appearance of the skin of the target area, as well as thicken the skin, tighten the skin, improve skin laxity, and/or reduce discoloration of the skin.

In one embodiment, the radiation can stimulate a heat-shock response in P. acnes, a bacteria that can cause acne and acne lesions. The heat-shock response can damage or destroy the P. acnes, reducing inflammation and improving the appearance of the skin or a lesion.

A variety of methods useful in measuring sebum production and useful in the practice of the invention are thoroughly documented in the art. For example, the level of sebum production can be measured by using commercially available sebutape or by means of a sebumeter.

Sebutape is a microporous patch available from CeDerm Corporation (17430 Campbell Rd., Dallas, Tex. 75252). Sebutape detects sebum production without the use of any solvents, powders, or chemicals. The microporous patch acts as a passive collector of sebum. Gradual displacement of air in the pores of the patch changes the patches appearance. The sebum filled pores in the patch do not scatter light and thus appear transparent. The size of the transparent area is a measure of the amount of sebum collected. Patches can be placed on a dark background storage card for evaluation by eye or by computer imaging (Elsner (1995) in “BIOENGINEERING OF THE SKIN: METHODS AND INSTRUMENTATION,” Berardesca, et al., eds., 81-89, CRC Press, Boca Raton, Fla.).

In addition to sebutape, sebum production can be measured by means of a device referred to in the art as a sebumeter, for example, a model SM 810 PC sebumeter, available from Courage & Khazaka (Mathias-Bruggen Str. 91, Koln, Germany). A sebumeter measures the content of sebum in the stratum corneum of skin, the values of which are expressed in micrograms/cm². The sebumeter can be fitted with a manual data collector which has a band designed to absorb skin sebum. The band is 0.1 mm thick and has a 64 mm² contact surface. The higher the amount of lipids present in the band, the higher the film transparency. The numeric values shown on the display are directly proportional to the band transparency and thereby to the amount of lipids present in the band itself (Elsner (1995) supra and http://www.corage-khazaka.de/products.htm and Clarys and Barel (1995) Quantitative Evaluation of Skin Surface Lipids, CLINICS IN DERMATOLOGY 13: 307-321). Heating of the dermal region may be accomplished by applying to the skin any light source capable of heating living tissue to a depth where sebaceous follicles are located. Heating energy can be provided by coherent light or incoherent light. Coherent light sources useful in the practice of the invention include, but are not limited to, pulsed, scanned or gated CW lasers. Light sources can also include a single laser chip, a LED bank, or collimating optics.

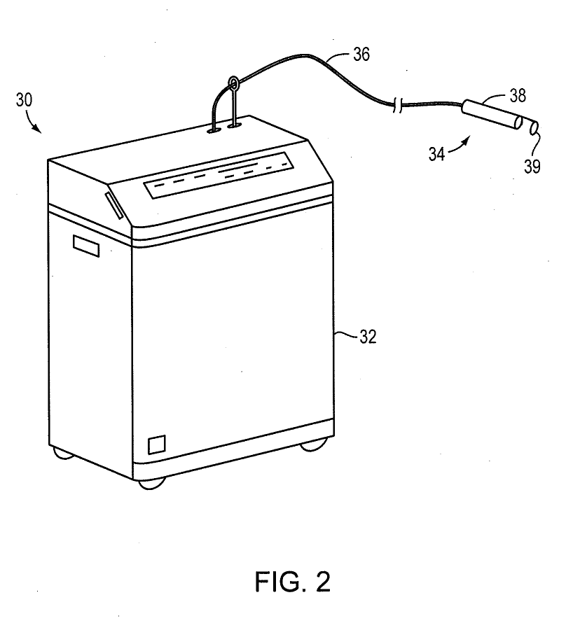

FIG. 2 shows an exemplary embodiment of a system 30 for treating tissue. The system 30 can be used to non-invasively deliver a radiation to a target area. For example, the radiation can be delivered through an external surface of skin over the target area. The system 30 includes an energy source 32 and a delivery system 34. In one embodiment, a radiation provided by the energy source 32 is directed via the delivery system 34 to a target area. In the illustrated embodiment, the delivery system 34 includes a fiber 36 having a circular cross-section and a handpiece 38. A radiation can be delivered by the fiber 36 to the handpiece 38, which can include an optical system (e.g., an optic or system of optics) to direct the radiation to the target area. A user can hold or manipulate the handpiece 38 to irradiate the target area. The handpiece 38 can be positioned in contact with a skin surface, can be positioned adjacent a skin surface, can be positioned proximate a skin surface, can be positioned spaced from a skin surface, or a combination of the aforementioned. In the embodiment shown, the handpiece 38 includes a spacer 39 to space the handpiece 38 from the skin surface. In one embodiment, the spacer 39 can be a distance gauge, which can aid a practitioner with placement of the handpiece 38.

In various embodiments, the energy source 32 can be at least one of an incoherent light source, a coherent light source, a microwave generator, a radio-frequency (“RF”) generator, and ultrasound radiation generator. In one embodiment, the source generates ultrasonic energy that is used to treat the tissue. In some embodiments, two or more sources can be used together to effect a treatment. For example, an incoherent source can be used to provide a first radiation while a coherent source provides a second radiation. The first and second beams of radiation can share a common wavelength or can have different wavelengths. In an embodiment using an incoherent light source or a coherent light source, the radiation can be a pulsed beam, a scanned beam, or a gated continuous wave (CW) beam. The delivery system 34 can include a cooling apparatus for cooling an exposed surface of skin before, during, or after treatment.

In another embodiment, the light used to thermally injure the sebaceous glands and/or the dermal tissue can originate from a compact, handheld device including a diode laser alone or in combination with additional apparatus such as an optical fiber, doped in such a way so as to delivery energy at a wavelength and power level so as to be therapeutically effective.

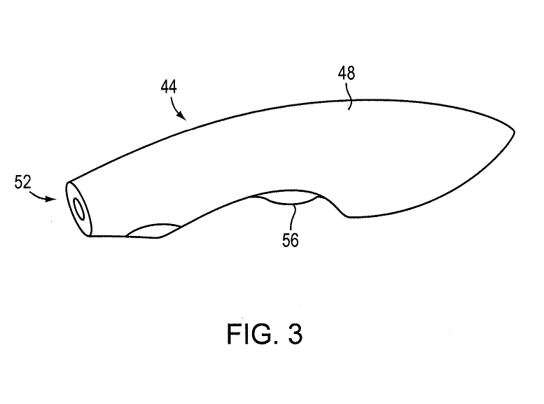

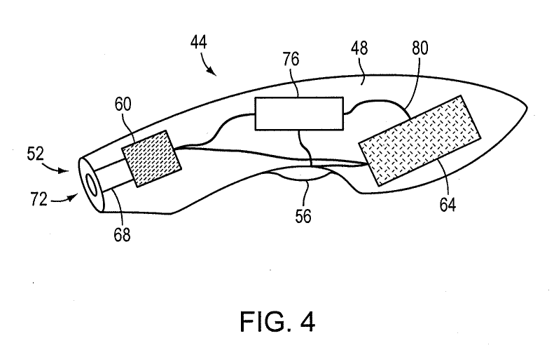

FIG. 3 shows an exemplary embodiment of a compact, handheld device 44 for treating tissue. The device 44 includes a handheld housing 48, a power source (shown in FIG. 4) associated with the handheld housing 48, an emitter portion 52 disposed at a first end of the handheld housing 48, and an activator 56 associated with the handheld housing 48 for activating the emitter portion 52. In one embodiment, the device 44 can include a disposable tip.

FIG. 4 shows a section view of the compact, handheld device 44. The emitter portion 52 includes an energy source 60. The emitter portion 52 or the energy source 60 receives power from a power source 64 to generate a radiation 68, which can exit an aperture 72 of the handheld housing 48. The device 44 can include a microprocessor 76 for controlling output of the device 44. The emitter portion 52, the activator 56, the power source 64, and the microprocessor 76 can be connected by electrical connectors 80, such as wires, although wireless communication can also be used. In one embodiment, the emitter portion 52, the power source 64, and the microprocessor 76 are disposed inside the handheld housing 48 to form the compact, handheld device 44.

The compact, handheld device 44 can be sized to fit into a user's hand for treatment of a sebaceous follicle disorder. The device 44 can be sized to fit into a user's pocket, e.g., for ease of transport. The device 44 shown in FIGS. 3 and 4 is oblong, although other shapes can be used as well. In one embodiment, the device 44 is about 3 inches in length and about 0.75 inch in diameter at the first end of the handheld housing 48. The device 44 can be lightweight, weighing about 0.25 lb to 5 lbs. The ergonomics of the device 44 can be such that it fits comfortably into a user's hand. The device 44 can include a gripping mechanism or grooves for fingers or a thumb.

The power source 64 can be a battery or plug adaptor connectable with a wall outlet. The power source can be rechargeable or disposable. In some embodiments, the power source 64 can be adapted to receive solar energy. In certain embodiments, the output voltage of the power source 64 can be between about 1.5 V and about 50 V. In some embodiments, the power source 64 can be less than about 25 V. In one embodiment, the power source 64 is a 9V battery. In one embodiment, the power source 64 can have a shelf life comparable to that of the energy source 60.

In one embodiment, the battery is flexible and can conform to the inside of the handheld housing 48. Power Paper Ltd. (Tel Aviv, Israel) manufactures one suitable battery. The chemicals used in Power Paper's battery are a combination of zinc and manganese dioxide. The battery may be printed using silkscreen technology onto almost any surface, including paper or flexible plastic. In one embodiment, the power source 64 includes five printed layers including collectors on the top and the bottom, an anode, a cathode, and an electrolyte core. A one-square-inch printed layer battery can provides 1.5 V for 15 mAh, is about 0.5 mm thick, and has a shelf life of up to about two and a half years.

The energy source 60 can be diode laser or other compact source. Exemplary sources are described in more detail below. In some embodiment, the energy source can target fat relative to water, or water relative to fat. In one embodiment, the energy source 60 can include a first laser diode doped to provide a first radiation having a wavelength preferentially absorbed by fat. The energy source 60 can include a second laser diode doped to provide a second radiation having a wavelength preferentially absorbed by water. The microprocessor 76 can control the duration of irradiation of a target region of skin, the intensity or fluence of the radiation, and/or the wavelength of the radiation. The microprocessor 76 can include a memory medium programmed to activate the emitter portion 52 or the energy source 60 for a preselected duration of time. For example, the energy source 60 can deliver about 20 J of energy for between about 3 seconds and 10 seconds. In one embodiment, a camera flash is used. The camera flash can be adapted to deliver the radiation in a pulse of about 100 μs. In one embodiment, the microprocessor 76 can include a timing circuit configured to activate the emitter portion 52 or the energy source 60 for a preselected duration of time.

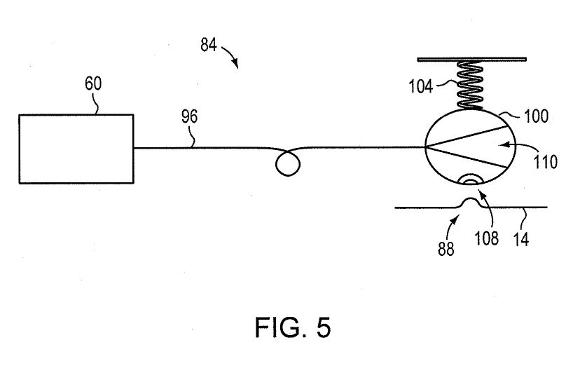

FIG. 5 shows another embodiment of a device 84 for treating a sebaceous follicle disorder 88 of skin 14. The device 84 includes an energy source 60 and an optical fiber 96 delivering output of the energy source 60 to a diffusing optic 100. An interlock mechanism 104 can be used to ensure that radiation is delivered when the device 84 is in contact with skin 92. The interlock mechanism 104 can be a pressure switch activated when an aperture 108 is over or surrounding the disorder 88. The aperture 108 can be between about 3 mm and about 5 mm, although larger or smaller apertures can be used depending on the application. In general, the interlock mechanism 104, or at least one switch, can be adapted to prevent emission of the radiation until the handpiece is substantially adjacent a surface of the skin.

The interlock mechanism 104 can be activated by pressure from a user or from pressure when the device is pressed against the skin 14. The diffusing optic 100 can be an integrating sphere. In various embodiments, the diffusing optic 100 can be a lens, a filter, a prism, a diffusion screen, a ground glass diffusion optic, an integrating sphere, a diverging lens, or some combination of the aforementioned. The diffusing optic 100 can prevent a user from injuring themselves, for example, by injuring a retina or overheating a portion of skin either by direct heating or an inadvertent reflection. The diffusing optic 100 can reduce the spatial coherence of the treatment beam. The diffusing optic 100 can scatter radiation 110 exiting the optical fiber 96. In one embodiment, the energy source 60 can be an eye safe diode laser, e.g., operating between about 1.4 μm and about 2.2 μm.

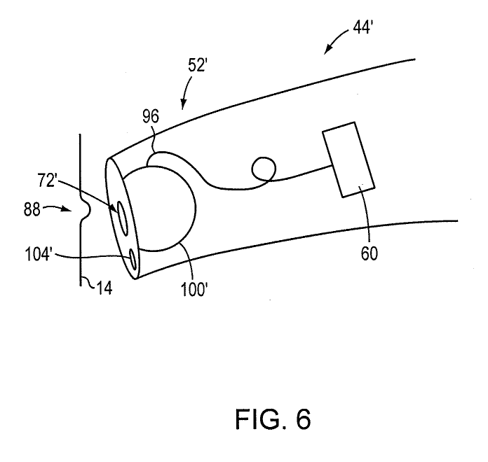

FIG. 6 shows an embodiment of an emitter portion 52′ of a compact, handheld device 44′ including a diffusing optic 100′ and an interlock mechanism 104′. The device 44′ can be used for treating a sebaceous follicle disorder 88 of skin 14. The energy source 60 is coupled to the diffusing optic 100′ using optical fiber 80. The aperture 72′ of the emitter portion 52′ can be aligned with the aperture of the diffusing optic 100′.

In the embodiments described above, the parameter ranges for the radiation can be selected to cause thermal injury to the sebaceous glands and/or to portions of the dermis where the sebaceous glands typically are present while at the same time avoiding injury to the epidermis and surrounding dermal regions. In particular, the wavelength of the radiation can be chosen to maximize absorption by the targeted region of the dermis, and the fluence or power density, depending on the type of radiation, chosen to minimize treatment related side-effects, including, for example, erythema, hypopigmentation, hyperpigmentation, and/or edema.

To target regions of the skin containing sebaceous follicles, it is desirable to use light that can penetrate the skin to depths up to about 2,000 microns. In certain embodiments, it is desirable to use light that can penetrate the skin to depths up to about in the range of values from about 200 microns to about 1,000 microns. It is understood that the depth of penetration of light of a given wavelength depends on the absorption and scattering properties of a tissue of interest. By selecting appropriate parameters, it is possible to target selected zones within the dermis of the skin.

In the visible to near infra-red region of the electromagnetic spectrum, absorption by hemoglobin and melanin contribute to the absorption properties of tissue, whereas in the near infra-red to far infra-red regions of the electromagnetic spectrum, absorption by water, fat, and fatty tissue contribute significantly to the absorption properties of tissue. Fat and/or fatty tissue can include sebum, sebocytes, sebum producing glands, such as a sebaceous gland, lipid containing tissue, or other dermal or epidermal tissue including fat. In the near to far infra-red regions of the electromagnetic spectrum, the light provided can have a wavelength that has a water absorption coefficient value in the range of about 0.1 cm⁻¹ to about 200 cm⁻¹, although tissue with a larger or smaller water absorption coefficient can be targeted depending on the application. In certain embodiments, tissue can be targeted with a fatty tissue absorption coefficient value in the range of about 0.01 cm⁻¹ to about 100 cm⁻¹, although tissue with a larger or smaller water absorption coefficient can be targeted depending on the application.

Certain wavelengths can be chosen to preferentially target water rather than fat and/or fatty tissue in a patient's skin, while other wavelengths can be selected to preferentially target fat and/or fatty tissue rather than water. In some embodiments, a wavelength can be chosen that does not preferentially target any of water, fat, or fatty tissue. In one embodiment, a wavelength that preferentially targets fat and/or fatty tissue rather than water can have a wavelength for which the ratio of the tissue absorption coefficient for fat to the tissue absorption coefficient for water is about or greater than 0.5. (See, e.g., U.S. Pat. No. 6,605,080, the entire disclosure of which is incorporated by reference in its entirety.) In some embodiments, a chromophore can be applied to the targeted region of the skin, and the radiation can be selected to be absorbed by the chromophore.

In various embodiments, the light can have a wavelength in the range from about 400 nm to about 2,600 nm, although longer and shorter wavelengths can be used depending on the application. In some embodiments, the wavelength can be between about 1,160 nm and about 2,200 nm. In one detailed embodiment, the wavelength of the radiation can be about 1,208 nm, 1,270 nm, 1,310 nm, 1,450 nm, 1,550 nm, 1,720 nm, 1,930 nm, or 2,100 mm.

In certain embodiments, the wavelength can be between 0.95 to 1.16 microns, more preferably from 0.97 to 1.15 microns, and more preferably from 1.00 to 1.10 microns. In another embodiment, the light has a wavelength in the range from 1.30 to 1.65 microns, more preferably from 1.32 to 1.60 microns, from 1.37 to 1.55 microns, and most preferably from 1.40 to 1.55 microns. In another embodiment, the light has a wavelength in the range form 1.85 to 2.38 microns, more preferably from 1.85 to 2.20 microns, more preferably from 1.90 to 2.15 microns, and most preferably from 1.91 to 2.10 microns. At these wavelengths or wavelength regions, the light is absorbed more preferentially by water than by fat and/or fatty tissue in the skin.

In certain embodiments, the wavelength can be between about 1,190 nm and about 1230 nm, about 1,700 nm and about 1,760 nm, or about 2,280 nm and about 2,350 nm. In one embodiment, the wavelength is about 1,210 nm. In one embodiment, the wavelength is about 1,720 nm. At these wavelengths or wavelength regions, the light is absorbed more preferentially by fat and/or fatty tissue than by water in the skin.

Exemplary laser sources that can be used to treat a sebaceous follicle disorder include, for example, a 1.06 micron Nd:YAG laser, a 1.15 micron helium neon laser, a 1.33 micron Nd:YAG laser, a 1.39 micron Raman shifted Nd:YAG laser, a 1.45 micron diode laser, a 1.48 micron diode laser, a 1.54 micron Er:Glass laser, a 1.54 micron Raman shifted Nd:YAG laser, a 1.57 micron Nd:YAG laser, a 1.91 micron Raman shifted Nd:YAG laser, a 2.10 micron Ho:YAG laser, or another diode laser with appropriate substrate and doping. For example, diode lasers can be formed having an output of about 1,210 nm or about 1,700 nm. The light may be pulsed, scanned or gated continuous wave laser radiation. Suitable incoherent radiation sources include black body radiation sources (e.g., a heated wire), filament lamps (e.g., a tungsten filament lamp), arc lamps, and flashlamps (e.g., a flashlamp including a spectral filter).

In various embodiments, the radiation can have a fluence between about 0.1 J/cm² and about 500 J/cm², although higher and lower fluences can be used depending on the application. In some embodiments, the fluence can be between about 1 J/cm² and about 100 J/cm². In one embodiment, the fluence is between about 3 J/cm² and about 20 J/cm². In various embodiments, the radiation can have a power density between about 0.1 W/cm² and about 10,000 W/cm², although higher and lower power densities can be used depending on the application. In some embodiments, the radiation can have a power density between about 1 W/cm² and about 10 W/cm².

In various embodiments, the radiation can have a spotsize between about 0.5 mm and about 25 mm, although larger and smaller spotsizes can be used depending on the application. In one embodiment, the radiation can have a spotsize of about 10 mm. In various embodiments, the radiation can have a pulse duration between about 10 μs and about 30 s, although larger and smaller pulse durations can be used depending on the application. In some embodiments, the radiation can have a pulse duration between about 0.1 seconds and about 20 seconds.

In various embodiments, the radiation can be delivered at a rate of between about 0.1 pulse per second and about 10 pulses per second, although faster and slower pulse rates can be used depending on the application.

In various embodiments, the parameters of the radiation can be selected to deliver the radiation to a predetermined depth, e.g., up to about 2 mm. In some embodiments, the radiation can be delivered to the target area about 0.1 mm to about 2 mm below an exposed surface of the skin, although shallower or deeper depths can be selected depending on the application. In one embodiment, the radiation is delivered to the target area about 1 mm to about 10 mm below an exposed surface of the skin.

In various embodiments, the radiation can converge with a predetermined focus in the target area below an exposed surface of the skin. In one embodiment, the radiation converges with a focus in the target area about 1 mm below an exposed surface of skin, although shallower or deeper depths can be selected depending on the application. To minimize unwanted thermal injury to tissue not targeted (e.g., an exposed surface of the target area and/or the epidermal layer), the delivery system 34 shown in FIG. 2 can include a cooling system for cooling before, during or after delivery of radiation, or a combination of the aforementioned. Cooling can include contact conduction cooling, bulk cooling, evaporative spray cooling, convective air flow cooling, thermoelectric cooling, or a combination of the aforementioned. Bulk cooling can include placing an ice pack or cold water on or over the skin prior to and/or after application of the radiation. In home or clinical applications, cooling can include contacting the skin with ice or an object cooled to a temperature below a temperature of the skin. In home or clinical applications, cooling can also include contacting the skin with a cooling solution or gel, such as those including acetone, alcohol, a compound suited to cool evaporatively, or an aqueous based gel.

In one embodiment, the handpiece 38 includes a skin contacting portion that can be brought into contact with the skin. The skin contacting portion can include a sapphire or glass window. The window can be cooled prior to applying the skin contacting portion to the skin. In various embodiments, the window can be cooled by refrigeration, by placing the window against a cold surface (e.g., ice), by dipping the window in a cold liquid (e.g., a cup of ice water), by holding the window under a flow of cold liquid (e.g., tap water), or by a combination of the aforementioned. In one embodiment, the skin contacting portion includes a fluid passage containing a cooling fluid. The cooling fluid can be a fluorocarbon type cooling fluid, which can be transparent to the radiation used. The cooling fluid can circulate through the fluid passage and past the window to cool the skin.

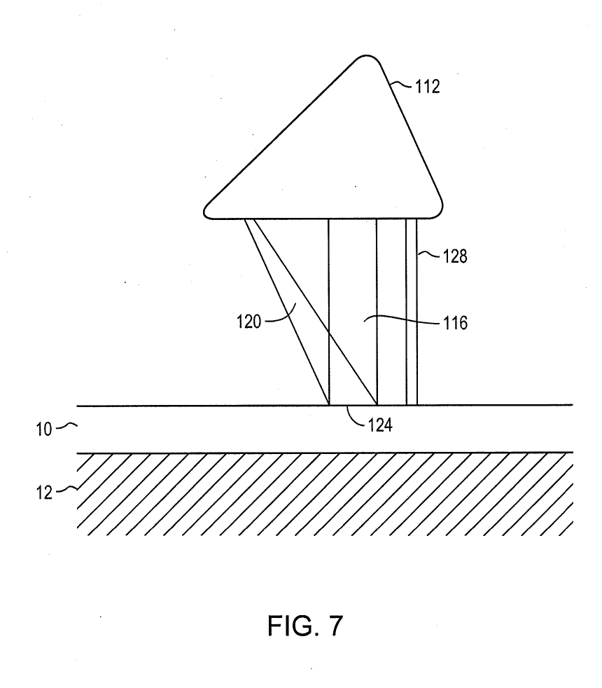

A spray cooling device can use cryogen, water, or air as a coolant. In one embodiment, a dynamic cooling device can be used to cool the skin (e.g., a DCD available from Candela Corporation). For example, the delivery system 34 shown in FIG. 2 can include tubing for delivering a cooling fluid to the handpiece 38. The tubing can be connected to a container of a low boiling point fluid, and the handpiece can include a valve for delivering a spurt of the fluid to the skin. Heat can be extracted from the skin by virtue of evaporative cooling of the low boiling point fluid. The fluid can be a non-toxic substance with high vapor pressure at normal body temperature, such as a Freon or tetrafluoroethane. Operation of such an embodiment is shown schematically in FIG. 7. Briefly, hand piece 112 is used to apply a radiation 116 from a laser source and a cryogen spray 120 to preselected region 124 of the skin surface. Application of the heat energy together with surface cooling cause thermal injury to the sebaceous follicle containing portion of the dermis while preserving epidermis 10. Guide 128 ensures that the handpiece 112 is positioned at the appropriate height above the surface of the skin to ensure that the radiation 116 and the cryogen spray 120 both contact skin surface at the preselected region 124.

The preselected region can be cooled prior to, contemporaneous with, and even after the application of the energy. The relative timing of cooling the skin surface and the application of heating energy depends, in part, on the depth to which thermal injury is to be prevented. Longer periods of cooling prior to the application of radiation allow more time for heat to diffuse out of the tissue and cause a thicker layer of tissue to be cooled, as compared to the thickness of the layer cooled by a short period of cooling. This thicker layer of cooled tissue sustains less thermal injury when the heating energy is subsequently applied. Continued cooling of the skin surface during the delivery of heating energy extracts heat from the upper layers of the skin as heat is deposited, thereby further protecting the upper skin layers (e.g., epidermis and dermis overlaying the target region) from thermal injury.

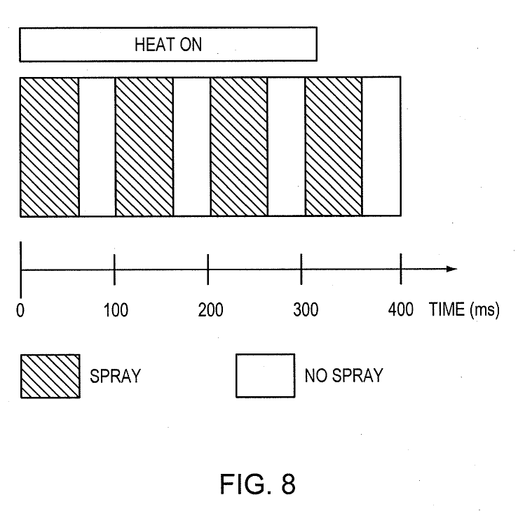

FIG. 8 provides an exemplary timing diagram showing time phases for the heating and/or cooling of the skin tissue afflicted with the disorder. The heating phase, represented by the horizontal bar, has a duration of 300 ms. Cooling, represented by vertical bars, comprises four separate cycles having a duration of 100 ms, each cycle comprising a 70 ms period when cryogen spray is applied to the skin surface and a 30 ms period when no cryogen spray is applied to the skin surface. In this timing diagram, the skin surface is cooled both (i) at the same time (i.e., the 70 ms phases of the first three cooling cycles) as the skin is exposed to the radiation and (ii) after (i.e., the 70 ms phase of the fourth cooling cycle) the skin has been exposed to the radiation.

Another exemplary timing scheme that may be used in the practice of the invention is similar to the previous scheme except that the light energy is provided intermittently with cooling steps in-between each of the heating steps. For example, an exemplary scheme may include a pre-laser application of coolant, a first laser pulse, an intervening application of coolant, a second laser pulse, an intervening application of coolant, a third laser pulse, an intervening application of coolant, a fourth laser pulse, and finally a post-laser application of coolant. In this type of scheme, the laser pulses can have the same or different durations. In a preferred scheme, the laser fluence ranges from about 8 to 24 J/cm² at a wavelength of 1450 nm. The total laser duration is 210 ms which is divided into four pulses of equal durations with three spurts of cryogen spray interspersed between the four laser pulses. In addition, there is a pre-laser spray and a post-laser spray. A 1450 nm laser and DCD system, Smoothbeam® is available from Candela Corporation and can be used in the practice of the invention. The laser provides a maximum output power on the skin of 15 W. Using such a device with a pulse duration of 210 ms, a maximum fluence of 25 J/cm can be achieved with a 4 mm circular spot at a repetition rate of 1 Hz. In order to speed up treatment times, it may be desirable to use lasers with a spot size greater than 4 mm in diameter. This can be achieved if the power output of the laser is increased.

In another embodiment, the light delivery and cooling systems may comprise separate systems. The cooling system may comprise a container of a cold fluid. Cooling the surface of the skin can be accomplished by applying the cold fluid onto the skin which then extracts heat from the skin on contact. In such an embodiment, a light delivery system comprises, for example, a handpiece containing optics for directing, collimating or focusing the radiation onto the targeting region of the skin surface. The light can be carried from the energy source, for example, a laser, to the handpiece by, for example, an optically transparent fiber, for example, an optical fiber. Coolant from a separate reservoir can be applied to the surface of the targeted region. In this embodiment, coolant from the reservoir flows to a dispensing unit separate from the energy delivery system via tubing connecting the reservoir and the dispensing unit. The coolant, once dispensed, can be retained in situ on the surface of the targeted region by a ring, for example, a transparent ring, which can be attached to the energy delivery system.

Selective heating of dermal regions containing the sebaceous glands can be achieved by selecting the appropriate heating and cooling parameters. For example, by choosing the appropriate wavelength it is possible to selectively heat portions of the dermis to a desired depth. For example, it is estimated that light having a wavelength of 1000 nm penetrates to a depth of approximately 600 microns. Accordingly, it is contemplated that dermal tissue greater than 600 microns from the skin surface will not be subjected to such intense heating as the region within 600 microns of the skin surface. Furthermore, it is possible to prevent damage to the skin surface by applying the types of cooling discussed hereinabove. By choosing appropriate parameters for the heating and cooling steps it is possible to selectively heat and thus selectively damage particular zones (target regions) within the skin which may contain a sebaceous gland and/or an infundibulum of a sebaceous follicle. Specifically, by choosing the radiation wavelength, the timing of the surface cooling, the cooling temperature, the radiation fluence and/or the power density as described above, the depth, thickness and degree of thermal injury can be confined to a particular zone within the dermis. Optimization of the foregoing parameters can be used to selectively heat regions of the dermis containing sebaceous follicles, more preferably regions containing sebaceous glands, while at the same time substantially or completely sparing injury to overlying regions of epidermis and dermis as well as underlying layers of dermis.

In various embodiments, the targeted region of the dermis can be heated to a temperature of between about 50° C. and about 80° C., although higher and lower temperatures can be used depending on the application. In one embodiment, the temperature is between about 55° C. and about 70° C. This temperature rise can be sufficient to affect the structure and/or function of sebaceous follicles disposed within the targeted region of the dermis. Studies have indicated that temperatures of 60° C. and above may be sufficient to create thermal damage to skin (Weaver & Stoll (1969) AEROSPACE MED 40: 24). The cooling system on the other hand, preferably cools the area of the skin above the targeted dermal region to temperatures below about 60° C., more preferably to below 50° C. during application of the heating energy, thereby minimizing or avoiding collateral thermal damage to the epidermis.

In various embodiments, the delivery system 34 shown in FIG. 2 can include a focusing system for focusing the radiation below the surface of the skin in the target area to affect at least one fat cell. The focusing system can direct the radiation to the target area about 0.5 mm to about 10 mm below the exposed surface of the skin. In some embodiments, the delivery system 34 can include a lens, a planoconvex lens, or a plurality of lens to focus the radiation. The lens can be placed proximate to a target area. Vacuum can be applied to suck the target area of skin against the concave contact surface of the lens. A radiation passing through the lens is focused to at least one fat cell in the target area.

In various embodiments, the energy source 32 or 60 shown in FIGS. 2-6 can be a diode laser having sufficient power to affect one or more fat cells. An advantage of diode lasers is that they can be fabricated at specific wavelengths that target fatty tissue. A limitation, though, of many diode laser devices and solid state devices targeting fatty tissue is the inability to produce sufficient power at a desired wavelength to effectuate a successful treatment.

In one embodiment, a diode laser can be a high powered semiconductor laser (e.g., as described in U.S. Pat. No. 5,394,492, the entire disclosure of which is hereby incorporated by reference). In one embodiment, the source of radiation is a fiber coupled diode laser array. For example, an optical source of radiation can include a plurality of light sources (e.g., semiconductor laser diodes) each adapted to emit light from a surface thereof. A plurality of first optical fibers each can have one end thereof adjacent the light emitting surface of a separate one of the light sources so as to receive the light emitted therefrom. The other ends of the first optical fibers can be bundled together in closely spaced relation so as to effectively emit a single beam of light, which is a combination of the beams from all of the first optical fibers. A second optical fiber can have an end adjacent the other ends of the first optical fibers to receive the light emitted from the bundle of first optical fibers. The light from the bundled other ends of the first optical fibers can be directed into the second optical fiber. The first optical fiber can have a numerical aperture less than that of the second fiber. In one embodiment, the first optical fiber can have brightness (e.g., the light power divided by the square of the product of the fiber core diameter and numerical aperture) greater than that of the second fiber.

Provided below are approximate penetration depths of light having different wavelengths, as estimated using two different algorithms. Table 1 lists wavelength in nanometers versus appropriate penetration depth (δ) in micrometers estimated using the formula:
δ(λ)=1/μₜᵣ(λ)
wherein μₜᵣ(λ) is given by the formula,
μₜᵣ(λ)=μₐ(λ)+μₛ′(λ)
wherein μₜᵣ(λ) is the wavelength dependent total attenuation coefficient, μₐ(λ) is the absorption coefficient, and μₛ′(λ) is the reduced scattering coefficient defined as,
μₜᵣ(λ)=μₛ(λ)*(1−g(λ)

wherein μₜᵣ(λ) is the signal scattering coefficient and g(λ) is the scattering anisotropy factor. Values of μₛ(λ) and μₛ′(λ) were taken from Simpson et al. (1998) PHYS. MED. BIOL. 43(9):2465-78 and from measurements of water absorption for estimated typical skin hydration levels of between 60% and 80%. The numbers provided in Table 1 are estimates based upon use of the algorithm and assumptions outlined above. These numbers are meant to provide general guidance and it is understood that the values may vary depending upon the particular type of algorithm and assumptions being relied upon.

TABLE 1

| Wavelength (nm) | Penetration Depth (microns) |
|---|---|
| 600 | 317 |
| 650 | 339 |
| 700 | 391 |
| 750 | 437 |
| 800 | 487 |
| 850 | 530 |
| 900 | 572 |
| 950 | 602 |
| 1000 | 624 |
| 1320 | 888 |
| 1330 | 867 |
| 1450 | 326 |
| 1550 | 581 |
| 1600 | 681 |
| 1700 | 731 |
| 1800 | 622 |
| 1900 | 133 |
| 2000 | 178 |
| 2100 | 346 |
| 2200 | 440 |
| 2300 | 375 |
| 2380 | 263 |

Similarly, it is possible to estimate approximate penetration depth as a reciprocal of the effective attenuation coefficient, μ_{eff}, calculated from the following equation derived by the diffusion approximation as previously described (Diffusion theory of light transport, section 6.4.1.2, in Optical-Thermal Response of Laser-Irradiated Tissue, (1998) Star, W., eds., Welch A. J. and van Gemert, M. J. C., Plenum Press, New York):
μ_{eff}={3μₐ[μₐ+(1−g)μₛ]}^{1/2},

where μₐ is the absorption coefficient, μₛ is the scattering coefficient, and g is the anisotropy factor. The typical scattering coefficient and the anisotropy factors in the mid infra-red region have been reported to be 100 cm⁻¹ and 0.9, respectively (Lask G. P. et al. “Nonablative laser treatment of facial rhytides,” Proc. SPIE, 2970, p. 338-349, 1997). These values are approximations. Furthermore, the absorption of skin is assumed to be 70% of the value of the water absorption coefficient. Table 2 lists wavelength in nanometers versus approximate penetration depth (δ) in micrometers using this formula.

TABLE 2

| Wavelength (nm) | Penetration Depth (microns) |
|---|---|
| 1320 | 1533 |
| 1330 | 1370 |
| 1450 | 230 |
| 1550 | 518 |
| 1600 | 696 |
| 1700 | 813 |
| 1800 | 583 |
| 1900 | 83 |
| 2000 | 113 |
| 2100 | 247 |
| 2200 | 339 |
| 2300 | 274 |
| 2380 | 177 |

Although the method of the invention can treat sebaceous follicle disorders in the absence of an exogenously added energy absorbing material, under certain circumstances, it may be beneficial to introduce such a material into the targeted region prior to application of the heat energy. For example, where the energy source is coherent or incoherent radiation, an externally injected radiation absorber, for example, a non-toxic dye, for example, indocynanine green or methylene blue, can be injected into the targeted dermal region. A radiation source provides radiation which is absorbed by tissue containing the absorber. As a result, use of a radiation absorbing material in combination with surface cooling can confine thermal injury or damage to the targeted dermal regions thereby minimizing potential injury to surrounding tissue.

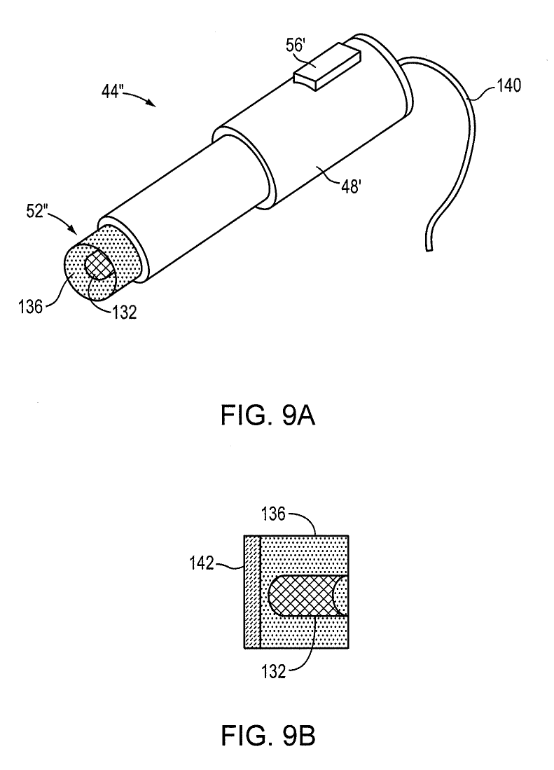

FIG. 9 shows another example of a compact, handheld device 44″ for treating tissue. The device 44″ includes a handheld housing 48′ with an emitter portion 52″ and an activator 56′. The emitter portion 52″ includes a lamp 132 and a reflector 136. The handheld housing 48′ includes a connector 140 to a power source. In other embodiments, the handheld housing 48′ can include an internal power source (not shown) or a directly attached power source (not shown).

The lamp 132 can be a tungsten filament lamp. In one detailed embodiment, the lamp 132 is a model MR3G-1089 available from Gilway Company (Woburn, Mass.). Suitable lamps include, but are not limited to, vacuum lamps, gas filled lamps (e.g., xenon, krypton, argon, or nitrogen filled lamps), or halogen lamps. The lamp 132 can be driven by a DC voltage power source or a battery. The power source can have a voltage of about 9 V or less.

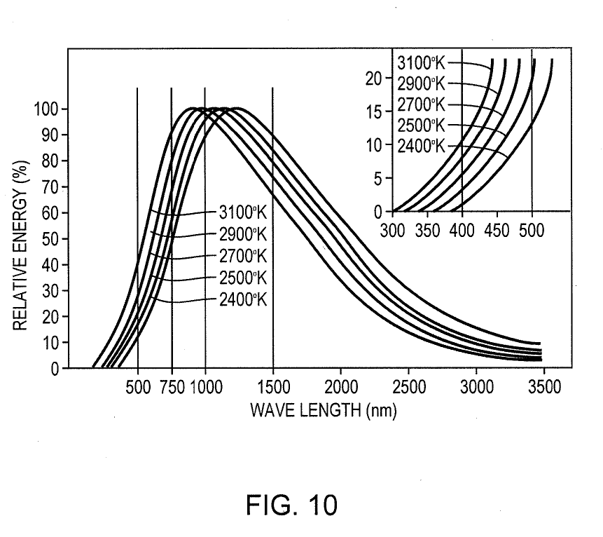

When heated (e.g., to about 2400 K), a tungsten filament lamp can produce infrared radiation characterized by a spectrum such as the spectrum shown in FIG. 10. The entire spectrum of radiation can be used to treat a sebaceous follicle disorder. Alternatively, a selected portion of the spectrum of radiation can be used to treat a sebaceous follicle disorder. A portion of the spectrum can be selected by passing the radiation through at least one of a bandpass filter, a high pass filter, and a low pass filter.

The lamp 132 can be placed inside the reflector 136, which can be an elliptical or parabolic reflector, to direct a radiation generated by the lamp 132 to the skin. For example, the reflector 136 can collect the radiation generated by the lamp 132 and deliver the radiation to a target region of skin to treat a sebaceous follicle disorder. The reflector 136 can be coated with optical material. For example, the reflector 136 can be coated with a reflective optical material such as gold, silver, or aluminum. A gold coating can increase infrared emission. In certain embodiments, the reflector 136 can be coated with optical material capable of filtering a desired wavelength or wavelengths (e.g., filtering a band of wavelengths).

The emitter portion 52″ can include a window 142, which can be positioned on an end of the reflector 136. The window 142 can contact the skin and preclude the skin from contacting the lamp 132 or the reflector 136. The window 142 can be transparent or translucent. In certain embodiments, the window 142 can be a filter. For example, the filter can pass selectively pass one or more wavelengths of energy to the skin (e.g., about 1,100 nm to 1,800 nm).

The window 142 can cool the skin surface. For example, the window 142 can act as a heat sink that removes thermal energy from the skin. In some embodiments, the window 142 can be actively cooled to enhance removal of thermal energy. The window 142 can be a portion of a conductive cooling device, as described above. Furthermore, the window 142 can be thermoelectric cooled and/or pre-cooled prior to contacting the skin. In some embodiments, the window 142 can be formed from sapphire or from glass. The window 132 can include a coating.

The device 44″ can be used to treat a region of skin of a diameter between about 0.3 cm to about 2 cm. In one embodiment, the diameter is about 1 cm. For example, the distal end of the device 44″ can be placed on a single acne lesion. The lamp 132 can be activated by pressing the activator 56′. In various embodiments, the radiation can be delivered to a target region of skin from about 1 second to about 200 seconds. In some embodiments, the radiation can be delivered to a target region of skin from about 5 seconds to about 100 seconds. In certain embodiments, the radiation can be delivered to a target region of skin from about 10 seconds to about 50 seconds. The device can be used to treat a sebaceous follicle disorder, treat acne lesions, and/or prevent acne lesions from appearing. A compact, handheld device is readily adapted to a higher power device with a larger lamp for treating larger regions of skin. For example, the device can be adapted to treat a region of skin of diameter greater than about 2 cm.

It can be advantageous to reduce both skin thickness and/or the presence of a fluid (e.g., blood and/or water) in target tissue, or in tissue interposed between the target tissue and the surface of the skin, by application of a vacuum to the skin. For example, a device can include a vacuum source and a skin contacting plate. The vacuum pressure results in a flattening of the skin against the plate, thus reducing skin thickness. Moreover, the flattening of the skin forces fluid contained in the tissue to the periphery of the device. A gel can be placed between the skin and a contacting plate to lubricate the skin. The gel can affect the strength of the vacuum. Generally, a system can apply suction to the skin, to effect at least one of reducing the thickness of at least one layer of the skin, reducing the amount of blood within at least one layer of the skin, reducing the amount of blood flow within at least one layer of the skin, and reducing the amount of pain or discomfort experienced by a patient. U.S. patent applications Ser. Nos. 11/057,542 and 11/401,674, the disclosures of which are incorporated herein in their entirety, discloses methods, apparatus, and vacuum elements that can be employed with the invention. A suitable device can be a pneumatic skin flattening (PSF™) device available from Inolase Ltd. (Israel).

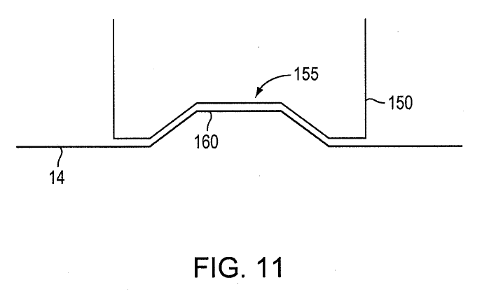

FIG. 11 shows a device including a skin contacting portion 150 of a delivery module that includes a vacuum device 155, which can cause an upward compression of a portion of the skin 14 such that region 160 is rendered substantially flat. This upward compression can reduce pain experienced by a patient during a treatment. The upward compression can also lead to expulsion of blood from the region 210 leading to more transparent skin. The expulsion of blood can substantially enhance the spectral selectivity and efficacy of the treatment. In certain embodiments, the skin contacting portion 150 is the window 142.

Stretching the skin can change the light scattering properties of the skin. Stretching the skin can make the skin “optically clear” by thinning a section of the skin and displacing chromophores in the skin, such as blood or other fluids in a target region or the tissue interposed between the target tissue and the surface of the skin. A relatively small change in the scattering properties of the skin can have a large effect on the amount of light reaching the target region. For example, to reduce lipid content in the skin, the target chromophores are those molecules residing deep in the skin tissue. Stretching the skin can enhance the light reaching the lipid and improve the efficiency of a treatment. The term “stretching” includes any physical modification to a region of skin resulting in an alteration in skin thickness or fluid content.

Stretching the skin can be performed by applying pressure to the skin. Pressure can result from pressing an object against the skin or applying a positive pressure (e.g., blowing air or other gas, or a fluid, or applying compression) or a negative pressure (e.g., applying vacuum or suction, as detailed herein). In certain embodiments, friction pads can be applied to the skin, and the friction pads can be laterally displaced in opposing directions to stretch the skin. By pulling the friction pads apart, the skin between the friction pads is stretched. By using friction pads, the skin can appear clear from the top. That is, there is no device or optic obstructing the path of the radiation. This is particularly advantageous if spray cooling is used.

A contact plate used for cooling the tissue as described herein can also be used to stretch the skin and thereby displace blood and other fluids in the contacted tissue section. Friction pads can be attached to or manufactured as part of a laser handpiece. When the handpiece is applied to the skin, the friction pads can be displaced to pull the skin away from the handpiece. The friction pads can be spring loaded. In certain embodiments, the handpiece can compress the skin, and the friction pads can stretch the skin.

A compact, handheld device having a diffusing unit can improve bodily safety during exposure to radiation by diverging the radiation with a diffuser. For example, at a first position of a distal end of a radiation source the energy density of the radiation can be substantially equal to the energy density of the radiation required for desired applications, and at a second position of the distal end the energy density of the emitted radiation can be significantly less than the energy density of the radiation. Accordingly, a device suitable for aesthetic treatment, medical treatment, or industrial treatment can be converted into an eye safe device. Eye safety can also be enhanced by measuring the radiance of the divergent radiation and issuing a warning as a result of a mishap if the radiance of the divergent radiation is greater than a predetermined safe value, and/or generating a visible flash prior to the emission of a pulse of radiation to induce an eye of a bystander to blink or to change its field of view in order to avoid the bystander staring at the radiation. U.S. patent application Ser. No. 11/229,983, the disclosure of which is incorporated herein in its entirety, discloses methods, apparatus, and diffusers that can be employed with the invention.

A compact, handheld device can include a handheld housing, a power source associated with the handheld housing, a radiation source, an integrating sphere, and an activator. The radiation source is disposed at a first end of the handheld housing and receives power from the power source to generate radiation. The integrating sphere improves bodily safety during exposure by at least one of scattering and multiple internal reflection of the radiation. The activator is associated with the handheld housing for activating the radiation source to generate the radiation, at least a portion of the radiation to be directed through the integrating sphere to a target region of skin to treat the sebaceous follicle disorder. The compact, handheld device can be used with an integrating sphere for treating a sebaceous follicle disorder.

In one embodiment, the compact, handheld device includes a single tip diode directly coupled into an integrating sphere. In another embodiment, the compact, handheld device includes a source of radiation coupled into an optical fiber, which is subsequently coupled into an integrating sphere. The source of radiation can be a single tip diode. The coupling angle can be about 90°. In some embodiments, the integrating sphere can induce rapid divergence of the radiation (e.g., the radiation can be eye safe at as little as about 1 mm). In certain embodiments, the integrating sphere includes a window 142 or skin contacting portion 150.

In various embodiments, a medicament can be applied to the skin for treating the sebaceous follicle disorder. The medicament can be applied at least one of before, during, and after treatment. In some embodiments, a kit includes a compact, handheld device generating radiation having energy in an amount sufficient to ameliorate the lesion and a medicament for treating the sebaceous follicle disorder at least one of before, during, and after applying radiation generated by the compact, handheld device. The medicament can include, but is not limited to, benzoyl peroxide, salicylic acid, sulfur, resorcinol, alcohol, and acetone.

In some embodiments, a sebaceous follicle disorder in a region of skin can be treated by (i) providing a compact, handheld device generating a first beam and a second radiation, the first beam and the second beam having sufficient energy to treat the sebaceous follicle disorder; (ii) delivering the first radiation at a first fluence to the region of skin in a first step to increase a temperature of the region to below about 60° C. to treat the sebaceous follicle disorder; and (iii) delivering the second radiation at a second fluence to the region of skin in a second step to maintain the temperature of the region below about 60° C. to treat the sebaceous follicle disorder.

In one embodiment, treating skin can reduce oiliness by (i) selecting a region of skin including at least one sebaceous gland; (ii) providing a compact, handheld device generating a radiation having sufficient energy to treat the sebaceous gland; and (iii) delivering the radiation to the region of skin to thermally effect at least one sebaceous gland and maintaining a temperature of the region of skin below about 60° C. to reduce skin oiliness.

In these and other embodiments, the invention can include delivering the radiation effects remodeling or opening of an infundibulum or a pore that carries sebum from the sebaceous gland to a surface of the skin. In various embodiments, the invention can include at least one of prophylaxis of sebaceous follicle disorders, reduction of skin oilyness, and reduction of the appearance of skin oilyness.

In various embodiments, the energy delivery system includes a skin contacting portion. The skin contacting portion can be transparent to the radiation, to allow the radiation to reach the skin. For example, the skin contacting portion can be sapphire or glass. The skin contacting portion can be heated to a temperature, for example 40-45° C., that is not high enough to cause a burn, but is higher than the normal skin temperature. The skin contacting portion can be placed in thermal contact with the skin for a pre-determined amount of time, to pre-heat the skin before delivering the radiation to the skin. The skin is then further heated by the radiation.

During pre-heating, heat is transferred from the skin contacting portion to the skin. During irradiation, when the skin temperature exceeds the skin contacting portion temperature, heat is transferred from the skin to the skin contacting portion. Thus, the skin contacting portion will act as a heat source in the beginning of the treatment and as a heat sink later in the treatment. When pre-heating is employed, less radiation may be required to bring the skin to the desired temperature, which can includes advantages such as the ability to use a lower power radiation source and/or a shorter treatment time. The skin contacting portion can be heated by methods including passing electric current through resistive meal lines placed on the skin contacting portion or blowing air at a desired temperature about the skin contacting portion.

In various embodiments, a hair removal treatment can be combined with a treatment. For example, the invention can include concurrent acne and hair removal treatments. In some embodiments, concurrent hair removal and treatment of hair removal related complications (e.g., ingrown hairs, razor bumps, pseudofolliculitis barbae, and infection) can be effected. In certain embodiments, a hair removal treatment can include affecting at least a melanin bearing portion of a follicle. In some embodiments, a hair removal treatment can be aided by the absorption of optical energy by blood vessels that surround or underlie hair follicles. U.S. patent applications Ser. Nos. 11/057,542, 11/229,983, and 11/401,674 disclose methods for hair removal, which can be adapted for use with the invention.

In various embodiments, a sensor can be used to detect and/or measure a reaction or a characteristic of the biological tissue. For example, a sensor can detect and/or measure at least one of a color and temperature of the skin. In one embodiment, biological tissue can be treated using radiation and biofeedback. A user is prompted for information relating to a biological tissue to be treated. The user is provided with one or more treatment parameters for the radiation based on the information. The user is prompted to trigger a device capable of emitting the radiation to treat the biological tissue. The method can be used iteratively. The parameters for subsequent radiation emissions can be modulated based upon user input, which can be derived from the biological tissue's reaction to the preceding radiation emission. Also, the method can facilitate user operation by providing an automated user interface. One advantage of the technology can be increased or complete automation of treatment.

A device can be used to treat biological tissue with the radiation and biofeedback. The apparatus includes a user interface, a processing unit, and a source of the radiation. The processing unit is coupled to the user interface and is configured to provide a signal to the user interface, to prompt a user for information relating to the biological tissue to be treated. The user interface is configured to provide a user input signal, including the information, to the processing unit. The source of the radiation is coupled to the processing unit. The processing unit is configured to provide a trigger signal to the source of the radiation, to cause the source to emit the radiation according to one or more treatment parameters based on the information of the user input signal.

### EQUIVALENTS

While the invention has been particularly shown and described with reference to specific embodiments, it should be understood by those skilled in the art that various changes in form and detail may be made therein without departing from the spirit and scope of the invention as defined by the appended claims.

### INCORPORATION BY REFERENCE

The content of each patent publication and scientific article identified hereinabove is expressly incorporated by reference herein in its entirely.

What is claimed is:

1. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion; and
delivering the radiation to the preselected dermal region of skin to ameliorate the lesion associated with the sebaceous follicle disorder while keeping the temperature of a region of the skin above the preselected dermal region below about 60° C. during application of the energy.

2. The method of claim 1 further comprising generating the radiation with at least one wavelength between about 1,200 nm and about 1,800 nm.

3. The method of claim 1 further comprising generating the radiation with at least one wavelength preferentially absorbed by fat relative to water.

4. The method of claim 1 wherein the at least one wavelength is between about 1,190 nm and about 1230 nm or between about 1,700 nm and about 1,760 nm.

5. The method of claim 1 wherein the compact, handheld device comprises a first laser diode doped to provide a first radiation having at least one wavelength preferentially absorbed by fat.

6. The method of claim 1 wherein the compact, handheld device further comprises a second laser diode doped to provide a second radiation having at least one wavelength preferentially absorbed by water.

7. The method of claim 1 wherein the compact, handheld device comprises a memory medium programmed to activate the device for a preselected duration of time.

8. The method of claim 1 wherein the radiation is delivered to the preselected dermal region of skin for about 100 μs to about 200 s.

9. A compact, handheld device for treating a sebaceous follicle disorder, the compact, handheld device comprising:
a handheld housing;
a power source associated with the handheld housing;
a tungsten lamp disposed at a first end of the handheld housing, the tungsten lamp receiving power from the power source to generate radiation;
an activator associated with the handheld housing for activating the tungsten lamp to generate the radiation;
a cooling device to keep the temperature of a region of the skin near the sebaceous follicle disorder below about 60° C. during application of the radiation; and
a reflector disposed proximally to the tungsten lamp, the reflector receiving at least a portion of the radiation generated by the tungsten lamp and directing at least a portion of the radiation to a target region of skin to treat the sebaceous follicle disorder.

10. The compact, handheld device of claim 9 wherein the radiation ameliorates a lesion associated with the sebaceous follicle disorder.

11. The compact, handheld device of claim 9 wherein the radiation has at least one wavelength between about 1,200 nm and about 1,800 nm.

12. The compact, handheld device of claim 9 wherein the radiation has at least one wavelength preferentially absorbed by fat relative to water.

13. The compact, handheld device of claim 12 wherein the at least one wavelength is between about 1,190 nm and about 1230 nm or between about 1,700 nm and about 1,760 nm.

14. The compact, handheld device of claim 9 wherein the radiation has a power density below about 10 W/cm².

15. The compact, handheld device of claim 9 wherein the power source comprises a battery.

16. The compact, handheld device of claim 9 wherein the power source is disposed inside the handheld housing.

17. The compact, handheld device of claim 9 further comprising a memory medium activated by the activator, the memory medium programmed to activate the emitter portion for a preselected duration of time.

18. The compact, handheld device of claim 9 further comprising an interlock switch to ensure radiation is only delivered when the device is in contact with the skin.

19. The compact, handheld device of claim 9 further comprising a timing circuit configured to activate the emitter for a preselected duration of time.

20. The compact, handheld device of claim 9 wherein the emitter portion comprises a filter to select a portion of the radiation to treat the sebaceous follicle disorder.

21. The compact, handheld device of claim 9 wherein the reflector includes a coating of an optically active material to enhance or filter emission of the radiation in a specified spectral window.

22. The compact, handheld device of claim 9 wherein the target region of skin is greater than about 1 cm in diameter.

23. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion, the compact, handheld device including a vacuum chamber transparent to the radiation;
drawing the preselected dermal region against a skin contacting element of the vacuum chamber; and
delivering the radiation to the preselected dermal region of skin to ameliorate the lesion associated with the sebaceous follicle disorder.

24. The method of claims 23 further comprising keeping the temperature of the region of the skin above the preselected dermal region below about 60° C. during application of the energy.

25. A compact, handheld device for treating a sebaceous follicle disorder, the compact, handheld device comprising:
a handheld housing;
a power source associated with the handheld housing;
a radiation source disposed at a first end of the handheld housing, the radiation source receiving power from the power source to generate radiation;
a vacuum chamber transparent to the radiation, the vacuum chamber for drawing the preselected dermal region against a skin contacting element of the vacuum chamber; and
an activator associated with the handheld housing for activating the radiation source to generate the radiation, at least a portion of the radiation to be directed through the vacuum chamber to a target region of skin to treat the sebaceous follicle disorder.

26. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion;
stretching the preselected dermal region of skin to enhance the penetration of the skin by the radiation; and
delivering the radiation to the preselected dermal region of skin to ameliorate the lesion associated with the sebaceous follicle disorder while keeping the temperature of the region of the skin above the preselected dermal region below about 60° C. during application of the energy.

27. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion, the compact, handheld device comprising a diffusing unit to improve bodily safety during exposure by scattering the radiation; and
delivering the scattered radiation to the preselected dermal region of skin to ameliorate the lesion associated with the sebaceous follicle disorder while keeping the temperature of the region of the skin above the preselected dermal region below about 60° C. during application of the energy.

28. A compact, handheld device for treating a sebaceous follicle disorder, the compact, handheld device comprising:
a handheld housing;
a power source associated with the handheld housing;
a radiation source disposed at a first end of the handheld housing, the radiation source receiving power from the power source to generate radiation;
a diffusing unit to improve bodily safety during exposure by scattering the radiation; and
an activator associated with the handheld housing for activating the radiation source to generate the radiation, at least a portion of the radiation to be directed through the diffusing unit to a target region of skin to treat the sebaceous follicle disorder.

29. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion, the compact, handheld device comprising an integrating sphere to improve bodily safety during exposure by at least one of scattering and multiple internal reflection of the radiation; and
delivering the scattered radiation to the preselected dermal region of skin to ameliorate the lesion associated with the sebaceous follicle disorder while keeping the temperature of the region of the skin above the preselected dermal region below about 60° C. during application of the energy.

30. A compact, handheld device for treating a sebaceous follicle disorder, the compact, handheld device comprising:
a handheld housing;
a power source associated with the handheld housing;
a radiation source disposed at a first end of the handheld housing, the radiation source receiving power from the power source to generate radiation;
an integrating sphere to improve bodily safety during exposure by at least one of scattering and multiple internal reflection of the radiation; and
an activator associated with the handheld housing for activating the radiation source to generate the radiation, at least a portion of the radiation to be directed through the integrating sphere to a target region of skin to treat the sebaceous follicle disorder.

31. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion;
delivering the radiation to the preselected dermal region of skin to ameliorate the lesion associated with the sebaceous follicle disorder; and
applying a medicament for treating the sebaceous follicle disorder at least one of before, during, and after treatment.

32. The method of claims 31 further comprising keeping the temperature of the region of the skin above the preselected dermal region below about 60° C. during application of the energy.

33. A kit for treating a sebaceous follicle disorder in a preselected dermal region of mammalian skin, the preselected dermal region having at least one lesion characteristic of the disorder disposed therein, the kit comprising:
a compact, handheld device generating radiation having energy in an amount sufficient to ameliorate the lesion; and
a medicament for treating the sebaceous follicle disorder at least one of before, during, and after applying radiation generated by the compact, handheld device.

34. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion;
applying a heat retaining element to a surface of the skin, the heat retaining element at least partially transparent to the radiation;
delivering a first portion of the radiation through the heat retaining element to the preselected dermal region of skin to ameliorate the lesion associated with the sebaceous follicle disorder; and
delivering a second portion of the radiation to the heat retaining element to convert the second portion of the radiation to thermal energy within the heat retaining element, which is subsequently conducted to the preselected dermal region of skin to facilitate amelioration of the lesion associated with the sebaceous follicle disorder.

35. The method of claim 34 further comprising at least one of increasing and maintaining for an extended period of time the temperature of the preselected dermal region of skin at below about 60° C.

36. A method of treating a sebaceous follicle disorder in a preselected dermal region of mammalian skin, the preselected dermal region having at least one lesion characteristic of the disorder disposed therein, the method comprising:
providing a compact, handheld device generating radiation having energy in an amount sufficient to ameliorate the lesion, the compact, handheld device comprising a skin contacting portion adapted for at least one of absorbing and releasing heat from the skin, the skin contacting portion substantially transparent to the radiation;
contacting the preselected dermal region with the skin contacting portion; and
delivering the radiation through the skin contacting portion to the preselected dermal region of skin to ameliorate the lesion associated with the sebaceous follicle disorder.

37. The method of claim 36 further comprising keeping the temperature of the region of the skin above the preselected dermal region below about 60° C. during application of the energy.

38. The method of claim 36 further comprising heating the skin contacting portion to a temperature above about 32° C. before contacting the preselected dermal region.

39. The method of claim 36 further comprising cooling the skin contacting portion to a temperature below about 32° C. before contacting the preselected dermal region.
